(19) United States
(12) Patent Application Publication
(10) Pub. No.: US 2005/0065503 A1
(43) Pub. Date: Mar. 24, 2005

(54) Method and apparatus for reducing the appearance of skin markings

(76) Inventors: Richard Anderson, Lexington, MA (US); Misbah Khan, Newport Beach, CA (US)

Correspondence Address: DORSEY & WHITNEY LLP;INTELLECTUAL PROPERTY DEPARTMENT, 250 PARK AVENUE, NEW YORK, NY, 10177, US

(21) Appl. No.: 10/921,524

(22) Filed: Aug. 19, 2004

Related U.S. Application Data
(60) Provisional application No. 60/496,120, filed on Aug. 19, 2003.
(60) Provisional application No. 60/496,126, filed on Aug. 19, 2003.
(60) Provisional application No. 60/496,128, filed on Aug. 19, 2003.

(51) Int. Cl. A61B 18/20

(52) U.S. Cl. 606/9

(57) ABSTRACT

Exemplary systems, apparatuses and methods are provided for performing a dermatological process to diminish the appearance of skin discoloration, in particular tattoos. For example, the arrangements implementing these systems may be specifically configured to produce particular radiation pulses that target phagocytic cells when skin of a subject is exposed to the particular radiation.

### CROSS REFERENCE TO RELATED APPLICATION(S)

This application claims priority from U.S. provisional application Nos. 60/496,120, 60/496,126 and 60/496,128, all filed on Aug. 19, 2003, the entire disclosures of which are incorporated herein by reference.

### FIELD OF THE INVENTION

The present invention relates to methods and apparatus which utilize electromagnetic radiation for a dermatological treatment and, more particularly to a method and apparatus that use optical radiation to damage a target area of skin surface for the dermatological treatment, in which such skin surface including a marking or discoloration.

### BACKGROUND INFORMATION

There has been an increasing demand for repair of or improvement to skin defects or marks, which can be induced by aging, sun exposure, dermatological diseases, traumatic effects, tattooing and the like. Such repair/improvement can be accomplished using a light source, such as a laser. Treatment modalities that involve light may generally depend on a thermal injury induced by a light source in a controlled manner. After thermal injury, the skin undergoes a complex wound healing response and natural repair of the injured area created by the light source.

The basic concept behind many laser biomedical applications is the theory of selective Photothermolysis, as described in R. Rox Anderson and J. A. Parrish, Selective Photothermolysis: Precise Microsurgery By Selective Absorption Of Pulsed Radiation, Science, vol. 222, pp. 524-527 (1983). This article describes, among other things, three primary concepts. The first concept is that light energy should be preferentially absorbed by the target in order to produce an effect. The second concept is that the fluence or energy per unit area delivered should be enough to produce a desired effect. The third concepts is that the radiant energy should be delivered to a target area in an appropriate amount of time, i.e., approximately equal to or less than the amount of time that it takes for the target to cool, often called the “thermal relaxation time”. Various techniques which may achieve this objective have been introduced in subsequent years. These techniques can be largely categorized in two groups for treating modalities/therapeutic applications: application of ablative lasers and application of non-ablative lasers. The ablative lasers tend to cause vaporization and heating of the skin in a controlled manner to a particular depth. These lasers are generally used for wrinkle removal and/or laser resurfacing. The non-ablative lasers target the structures inside the skin, and affect the target area in an extremely precise fashion without creating a significant amount of surrounding damage. Non-ablative lasers are used in the treatment of vascular lesions, i.e. port-wine stains, removal of hair, removal of tattoos, etc.

Laser resurfacing, sometimes referred to as ablative resurfacing, can be used for treating photo-damaged skin, scars, superficial pigmented lesions and superficial skin lesions. However, patients may experience major drawbacks after each laser resurfacing treatment, including pain, infection, scarring, edema, oozing, burning discomfort during first fourteen (14) days after treatment, skin discoloration, and possibly scarring as a subsequent complication. These ablative lasers (e.g. CO₂ and Er:YAG lasers) are not traditionally used for tattoo removal. This is because the tattoo ink is located deep inside the skin. Indeed, if the ablative lasers were to be used in a conventional manner to remove tattoo ink from the relevant depths within the skin, a much deeper tissue ablation would be required. However, such approaches almost always would lead to scarring and further complications, such as a thermal burn.

Generally, all conventional ablative laser treatments can result in some type of thermal skin damage to the treated area of the skin surface, including the epidermis and the dermis. The treatment with pulsed CO₂ or Er:YAG lasers is relatively aggressive and causes thermal skin damage to the epidermis and at least to the superficial dermis. Following treatment using CO₂ or Er:YAG lasers, a high incidence of complications occurs, including persistent erythema, hyperpigmentation, hypopigmentation, scarring, and infection (e.g., infection with bacteria or viruses such as Herpes simplex virus). These treatments are generally characterized by pulses of a high power laser scanned across the skin.

Lasers used for ablative purposes (e.g., CO₂ and Er:YAG lasers) are generally not used for tattoo removal for several reasons. However it is well known that ablation of tattooed skin with these lasers reliably removes the tattooed skin, leading to a scar. The tattoo ink may lie very deep in the skin (e.g., at a depth of approximately 1 mm), and remains resident within cells (e.g., fibroblasts) for many years at the location where the ink was originally introduced. In order for the lasers to ablate the skin containing the tattoo ink, the operator must ablate a relatively thick layer of skin, thus essentially creating a third degree burn at the target area. Such a treatment method creates a deep open wound that requires extensive post-operational care and management as part of healing such damaged area. In this procedure, even though a considerable portion of skin has been ablated, a residual portion of the tattoo ink remains in the area. Once treated, the skin is easily prone to infections and extensive scarring on a long-term basis. Additionally, the area of treatment of subjects having light-skinned complexions (e.g., Caucasians) tends to lose pigment after the healing process is complete, while the treatment area of the subjects having darker complexions tend to get darker and more heavily pigmented after the healing process. Thus, CO₂ and Er:YAG lasers are no longer frequently used to remove or lessen the appearance of tattoos.

In order to avoid the problems associated with ablative lasers, Q-switched lasers (e.g., Ruby laser, Alexandrite, Nd:YAG laser, and flash lamp pulsed dye laser) can be utilized. These lasers are generally tattoo color-dependent, in that they utilize various wavelengths for various colors, and target the ink particles contained within the cells situated deep within the skin. Such lasers usually operate at a very high power and fluences, and deliver a substantial amount of energy in a small fraction of a second (e.g., nano-seconds). The Q-switched lasers do not cause any ablation of the skin, and the surface of the skin generally stays intact. However, since the energy is delivered in extremely short pulses, stress waves and cavitation are likely generated around the tattoo particles so as to produce immediate whitening upon such laser exposure. This phenomenon is also responsible for creating lacunae or large spaces in the dermis, and causes the separation of the epidermis from the dermis at localized areas. In this manner, the cells containing the ink rupture and release the ink into the dermis.

Such laser treatments create a mechanism for disrupting the dermis containing the ink, and have a significantly lower risk of post-procedure complications as compared to the procedures that use the ablative lasers. Indeed, the utilization of Q-switched lasers for treatment of tattoos and other pigmented lesions of skin has become the industry standard. However, in order to obtain effective treatment the subject generally undergoes multiple treatments before improvement in a tattoo removal procedure is visualized. Typically, four to eight treatments are required to make the subject area of the skin either lighter and/or to obtain a significant removal of the tattoo. In certain cases (e.g., approximately 30% of the subjects), considerably more treatments (i.e. 10 or more treatments) will not be able to lighten tattoo to an acceptable level, and some tattoos respond little if at all (e.g., also approximately 30%). Since the risk of damaging the epidermis and non-tattooed structures of the dermis when the Q-switched lasers are used is much smaller than the risk with the use of the ablative lasers, the time needed for healing is minimal, typically about 1 week, and post-treatment care is simpler. The skin barrier function of the epidermis is better preserved and there is little risk of infection and scarring after typical tattoo treatments using non-ablative Q-switched lasers.

To perform the above-described procedures, Q-switched lasers are typically configured to have a pulse duration of between 5 and 100 ns with adjustable fluences. The important aspect of this treatment is Q-switched lasers do not remove the tattoo ink nor ablate the skin that contains them. The ink is released from the cells that contain them and is slowly removed from the dermis by the body's own response to this type of laser injury. Therefore, multiple (i.e. four to eight) treatments are required to lighten the tattoo satisfactorily. If the tattoo fails to respond, further treatments lead to increase risk of skin textural change and eventually scarring. Also, most Q-switched lasers are monochromatic, i.e., they can only emit energy having a particular bandwidth or color. The wavebands of the emissions of these lasers may be altered using frequency doubling or Raman shifting, however these techniques are imperfect and expensive. Therefore, in order to treat tattoos that come in multiple colors, more than one Q-switched laser is necessary to cover a large spectrum of colors to be treated. Additionally, there are no Q-switched lasers available to treat yellow, light blue, flesh toned and white tattoo inks.

Yet another problem encountered by the use of Q-switched lasers is their interaction with the natural pigment in the skin it self, called melanin. Successive treatments with Q-switched lasers can lead to loss of melanin, called hypopigmentation, in lighter skinned patients. On the other hand, darker skinned individuals can experience further darkening, called hyperpigmentation, of the site of treatment. Such consequences can cause certain patients to refrain from undergoing further treatments.

The advantage of tattoo treatment with these Q-switched lasers is that they target the tattoo ink particles contained within the cells, providing a more selective treatment. However, the effectiveness of treatment depends on light absorption by the inks, which is wavelength-dependent for different ink colors. For multi-colored tattoo, more than one type of Q-switched laser is often needed. The wavelength of the lasers is selective for a particular color and the pulse duration is extremely short, on the order of nano seconds, as it depends on the size of the particles (0-2 μm typically), which are the target. The tattoo ink particles heat up as they absorb energy from the laser light and eventually cause the cell containing such ink particles to rupture. The cells containing the ink particles rupture as well and release the ink into the dermis. After several laser treatments, the tattoo may lighten, but there is always ink remaining in the treated area.

Another problem with the traditional Q-switched lasers is that they do not cover the entire spectrum of colors that are so commonly used in body art. Colors like brown, light blue, orange and purple do not respond very well. Yet, there is no laser that can treat yellow, flesh toned or white colored tattoos. If the patient wishes to get rid of them, they have to undergo extensive surgeries and re-construction of the defect created by them.

Therefore, there is a need to provide a procedure and apparatus that effectively treats discoloration of the skin with minimum side effects, and avoids the deficiencies of the conventional procedures.

### SUMMARY OF THE INVENTION

It is therefore one of the objects of the present invention to provide an apparatus and method that effectively reduces the appearance of skin markings with minimal side effects. Another object of the present invention is to provide an apparatus and method that causes thermal skin damage to particular types of cells of the dermis, e.g. phagocytic cells, while sparing the epidermis to a large degree.

It is another object of the present invention to provide a system and method for treating skin conditions in which phagocytic cells of the dermis have ingested pigment particles, causing an unwanted pigmentation or coloration of the skin.

These and other objects can be achieved with the exemplary embodiment of the apparatus and method according to the present invention, in which a light emitting apparatus is provided. The apparatus includes a radiation generator that is configured to produce particular radiation pulses which target phagocytic cells when skin of a subject is exposed to the particular radiation.

In another advantageous embodiment of the present invention, an apparatus and method for decreasing the appearance of a tattoo on tattooed dermal tissue are provided. In this exemplary method, particular radiation is generated which has a fluence range between approximately 2 J/cm² and 20 J/cm² (or between approximately 2 J/cm² and 40 J/cm²), a spot-size diameter of the particular radiation beam of at least 3 mm, and a pulse width of between 1 μs and 300 μs in duration. In addition, the epidermal tissue of a subject is exposed to the particular radiation.

In yet another advantageous embodiment of the present invention, an apparatus and method for decreasing the appearance of a tattoo on a tattooed epidermal tissue are provided. In particular, particular radiation is generated having a fluence range between approximately 0.1 J/cm2 and 1 J/cm2, a spot-size diameter of the particular radiation beam of at least 3 mm, and a pulse width of between 10 μs and 1000 μs in duration.

In still another embodiment of the present invention, an apparatus and method for decreasing the appearance of a tattoo or tattooed skin are provided. In this exemplary method a plurality of radiation pulses are provided at a target area of tattooed skin, the plurality of radiation pulses are delivered sequentially at a rate of at least 1 Hz. In an aspect of the further embodiment, the target area may be cooled during delivery of the plurality of radiation pulses, to limit epidermal and dermal injury. In another aspect of the further embodiment, the target area may be cooled between one or more successive pulses during delivery of the plurality of radiation pulses.

In a further embodiment of the present invention, an apparatus and method for decreasing the appearance of a tattoo or tattooed skin are provided. The method including generating a plurality of radiation pulses specifically adapted to target phagocytic cells when the dermal tissue of a subject is exposed to the particular radiation, exposing the skin tissue of the subject to the radiation pulses at a particular frequency, determining whether the temperature of the skin exceeds a threshold value, and based on a result of the determining step, controlling the particular frequency.

### BRIEF DESCRIPTION OF THE DRAWINGS

For a more complete understanding of the present invention and its advantages, reference is now made to the following description, taken in conjunction with the accompanying drawings, in which:

FIG. 1 shows a first exemplary embodiment of a dermatological treatment system for conducting various treatments according to the present invention;

FIG. 2 shows a second exemplary embodiment of the dermatological treatment system for conducting various treatments according to the present invention;

FIG. 3 shows a cross-sectional view of skin that has been tattooed;

FIG. 4 shows a cross-sectional view of the skin following a traditional dermatological treatment using Q-switched lasers;

FIG. 5 shows a cross-sectional view of the skin following a dermatological treatment according to an exemplary embodiment of the present invention; and

FIG. 6 is a flow chart illustrating an exemplary embodiment of a dermatological process using electromagnetic radiation according to the present invention.

Throughout the drawings, the same reference numerals and characters, unless otherwise stated, are used to denote like features, elements, components, or portions of the illustrated embodiments. Moreover, while the present invention will now be described in detail with reference to the Figures, it is done so in connection with the illustrative embodiments.

### DETAILED DESCRIPTION OF THE PREFERRED EMBODIMENTS

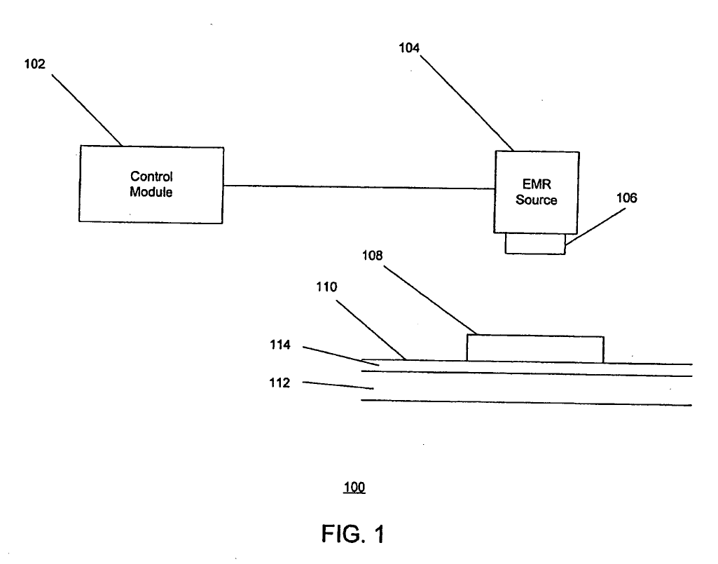

FIGS. 1, 2, 5 and 6 illustrate exemplary embodiments of methods and systems for dermatological treatment of a target area of skin. Generally, the exemplary methods and systems deliver an electromagnetic radiation to the patient's skin so as to induce thermal injury of dermal tissue of the skin, thus resulting in the reduction of skin markings. The skin markings may include tattoos, pigmented lesions, and the like. The pigmented lesions may include melasma, lentigines, and the like.

FIG. 1 illustrates a first exemplary embodiment of a dermatological treatment system 100 for conducting various dermatological treatments using electromagnetic radiation (“EMR”) to generate desired, target-selective photothermal skin damage of a target area according to the present invention. The system 100 may be used for a removal of unwanted pigment, a removal or reduction of the appearance of a tattoo, and/or similar dermatological applications. This system 100 can deliver EMR radiation to the skin surface that is tailored to specifically target phagocytic cells. As shown in FIG. 1, the system 100 includes a control module 102, an EMR source 104, delivery optics 106 and an optically transparent plate 108. The control module 102 is in communication with the EMR source 104, which in turn is operatively connected to the delivery optics 106.

In one exemplary variant of the first exemplary embodiment of the present invention, the control module 102 can be in wireless communication with the EMR source 104. In another variant, the control module 102 may be in wired communication with the EMR source 104. In still another variant, the EMR source 104 and the delivery optics 106 can be connected to the optically transparent plate 108.

The control module 102 can provide application specific settings to the EMR source 104. The EMR source 104 may receive these settings, and generate an EMR based on these settings. The settings can be used to control the wavelength of the EMR, the energy delivered to the skin, the power delivered to the skin, the pulse duration for each EMR pulse, the fluence of the EMR delivered to the skin, the number of EMR pulses, the delay between individual EMR pulses, the beam profile of the EMR, and the size of the area of the skin exposed to EMR. The energy produced by the EMR source 104 can be an optical radiation, which may be focused, collimated and/or directed by the delivery optics 106 to the optically transparent plate 108. The optically transparent plate 108 can be placed on a target area of a patient's skin 110, and can be actively cooled to minimize epidermal injury during treatment.

In another variant of the first exemplary embodiment of the present invention, the EMR source 104 may be laser, an arc lamp, a flashlamp, a laser diode array, the combination of each, and the like. In yet another exemplary embodiment, the EMR source 104 can be a ruby laser, an alexandrite laser, and/or a flashlamp pulsed dye laser. In still another variant of the first exemplary embodiment of the present invention, the EMR source 104 can be a Xenon flashlamp, a mixed gas flashlamp, a doped flashlamp and/or another intense pulsed light source.

Prior to being used in a dermatological treatment, the system 100 shown in FIG. 1 can be configured by a user. For example, the user may interface with the control module 102 in order to specify the specific settings usable for a particular procedure. For example, the user may specify the wavelength of the EMR, the energy delivered to the skin, the power delivered to the skin, the pulse duration for each EMR pulse, the fluence of the EMR delivered to the skin, the number of EMR pulses, the delay between individual EMR pulses, the beam profile of the EMR, and/or the size of the area of skin 110 exposed to EMR.

It should be understood that the settings can be specified by the characteristics of the beam generated by the EMR source 104 or the characteristics of the beam as it impinges the skin 110. For example, the beam may have one particular fluence magnitude at the source, and another fluence magnitude at the skin. The control system 102 can be configured to accept and utilize either setting from the user.

For a particular procedure according to the present invention, the EMR source 104 may be a laser. The EMR source 104 can be set to produce a substantially collimated pulsed EMR irradiation with various wavelengths. The EMR may be delivered to the skin in a substantially collimated beam, a divergent beam, or a highly divergent beam. A substantially collimated beam is typically produced when a laser is used. For removal of different colors of tattoo ink it is preferable to use different bandwidths. For example, “blue”, “green”, “red”, “infrared” and broadband red-near infrared wavebands can be used for the treatment of yellow, red, green/blue, and black inks, respectively. The “blue” waveband is approximately 420 nm-550 nm. The “green” waveband is approximately 500 nm-600 nm. The “red” waveband is approximately 620 nm-800 nm. The “infrared” waveband is approximately 700 nm-1200 nm. In addition, the broadband red-near infrared waveband is approximately 620 nm-1200 nm. In fair-skinned patients who have little melanin content in their epidermis, a broader range of wavelengths up to and including white light plus near-infrared, may be used without damaging the epidermis. Preferably, two wavebands may be utilized: the first waveband ranging from 600 nm to 1200 nm for treating black and green inks, and the second waveband ranging from 400 nm to 600 nm for treating red and yellow inks.

For use with the same or similar procedure, the EMR radiation may have a spectral bandwidth of at least 50 nm, but bandwidths of 100 nm to 500 nm or greater in width can also be utilized for a greater throughput. If a tattoo contains black ink, a spectral bandwidth of 800 nm or above may be used. The EMR source 104 produces the EMR in pulses. The length of these pulses, i.e., pulse width, may be between 1 μs and 1000 μs, and is preferably between 5 μs and 100 μs. The collimated pulsed EMR irradiation may be applied, which has a fluence between 0.1 J/cm² and 20 J/cm² (or between approximately 2 J/cm² and 40 J/cm²), preferably between 5 J/cm² and 10 J/cm² (or between approximately 5 J/cm² and 35 J/cm²), and a spot-size diameter of at least 3 mm (preferably at least 10 mm). The applied EMR should be able to achieve a temperature rise within the exposed areas of the skin which is at least sufficient to cause thermal damage to phagocytic cells in the dermis 112. The EMR source 104 may produce multiple pulses at a predetermined frequency. For example, the control module 102 may cause the EMR source 104 to produce these pulses at a frequency (i.e., pulse frequency) of between 1 Hz and 100 Hz, and preferably approximately at 10 Hz. The peak temperature sufficient to cause thermal damage in the exposed tissues is generally time dependant, and can be between 45° C. and 100° C. The peak temperature achieved in the phagocytic pigmented target cells of the dermis, and the average temperatures achieved in the bulk substance of the dermis surrounding these target cells, and anatomical depth of thermal damage can be adjusted by a selection of a particular wavelength, fluence per pulse, number of pulses, pulse repetition rate and skin surface cooling.

In an alternate embodiment of the present invention, three wavebands may be utilized. For example, the first waveband may have a range of 600 nm to 1200 nm for treating black and green inks, the second waveband may have a range of 400 nm to 550 nm for treating yellow inks, and the third waveband may have a range 500 nm to 600 nm for treating red inks.

In another exemplary embodiment, a light emitting apparatus can include a radiation generator producing radiation, or a beam of radiation, that affects phagocytic cells in a target portion of skin. The phagocytic cells include at least one of a particle of melanin and a particle of an exogenous artificial pigment. The radiation can thermally damage the phagocytic cells. In various embodiments, the radiation can have a wavelength of about 532 nm, of about 755 nm, or about 1064 nm. The radiation can have a pulse rate of between about 1 Hz and 5 Hz, and can have a pulse duration of between about 100 ms and about 120 ms. In some embodiments. the radiation can have a fluence between about 0.1 J/cm² and about 40 J/cm². In one detailed embodiment, the radiation generator can include a plurality of radiation sources, where each radiation source produces radiation with a different wavelength. For example, a first radiation source can produce radiation having a wavelength of about 532 nm and a second radiation source can have a wavelength of about 755 nm. A third radiation source can have a wavelength of about 1064 nm. Of course, other combination of wavelengths are possible in an apparatus including a plurality of radiation sources.

In another exemplary embodiment of the present invention, the EMR source 104 may be a flashlamp or another device capable of producing an intense pulsed light. The EMR source 104 may be set to produce a pulsed EMR irradiation with various wavelengths. The EMR may be delivered to the skin in a substantially collimated beam, a divergent beam, or a highly divergent beam. A highly divergent beam is typically produced when a flashlamp is used. Preferably, two wavebands may be utilized. For example, the first waveband may have a range of 600 nm to 1200 nm for treating black and green inks, and the second waveband may have a range of 400 nm to 600 nm for treating red and yellow inks. Other wavebands, mentioned above, could also be utilized depending on the particular application.

The EMR radiation should have a spectral bandwidth of at least 50 nm when a flashlamp is used, however, bandwidths of 100 nm to 500 nm can be utilized for greater throughput. The spectral bandwidth may be controlled by spectral filtering of a broader spectral output of the EMR source. Wavelength-converting filters, such as fluorescent filters which absorb short wavelengths and pre-emit this absorbed energy within the spectral band used for skin treatment, can also be used. The EMR source 104 may produce the EMR radiation in pulses. The length of these pulses, i.e., pulse width, may be between 10 μs and 1000 μs, preferably between 50 μs and 200 μs, and ideally approximately 100 μs. The pulsed EMR irradiation may be applied, which has a fluence between 0.1 J/cm² and 20 J/cm² (or between 0.1 J/cm² and 40 J/cm²), preferably between 0.1 J/cm² and 1 j/cm², and a spot-size diameter of at least 3 mm, preferably at least 5 mm. When a flashlamp is used, a train of pulses as defined above are delivered to a target area. The applied EMR should be able to achieve a temperature rise within the exposed areas of the skin that is at least sufficient to cause thermal damage to phagocytic cells in the dermis 112. The EMR source 104 may produce multiple pulses at a predetermined frequency. For example, the control module 102 may cause the EMR source 104 to produce these pulses at a frequency (i.e. pulse frequency) of between 1 Hz and 100 Hz, preferably between 2 Hz and 20 Hz. The peak temperature sufficient to cause thermal damage in the exposed tissues may be time dependant and in the range of 45° C. to 150° C. For the exposure times firmly in the range of 0.1 ms to 10 ms, the preferred minimum temperature rise for causing the thermal damage may be in the range of approximately 60° C. to 100° C. The depth of thermal damage can be adjusted by a selection of at least one of the wavelength, fluence per pulse, and number of pulses.

In an alternate embodiment of the present invention, three wavebands are utilized. For example, the first waveband can be 600 nm to 1200 nm for treating black and green inks, the second waveband can be 400 nm to 550 nm for treating yellow inks, and the third waveband may be 500 nm to 600 nm for treating red inks.

During an exemplary dermatological treatment, the system 100 may produce EMR which is directed to the target area of the skin 114. During the treatment, the temperature of the skin may be monitored and used to control the treatment parameters, e.g., pulse fluence and/or repetition rate. Skin temperature monitoring may be accomplished at the skin surface by a thermocouple in contact with the skin, thermocouple in an element of the device which is close to the skin, or a far-infrared detector which monitors black body emission from the skin surface. The EMR may be pulsed multiple times to create the appropriate effect and irradiation at the target area of the skin 114.

After the dermatological treatment is completed, certain portions of the target area of the skin 114 are damaged. Preferably, the epidermis 114 can be largely undamaged and the phagocytic cells of the dermis 112 are damaged. The epidermis 114 and other portions of the dermis 112 may also be damaged by the EMR.

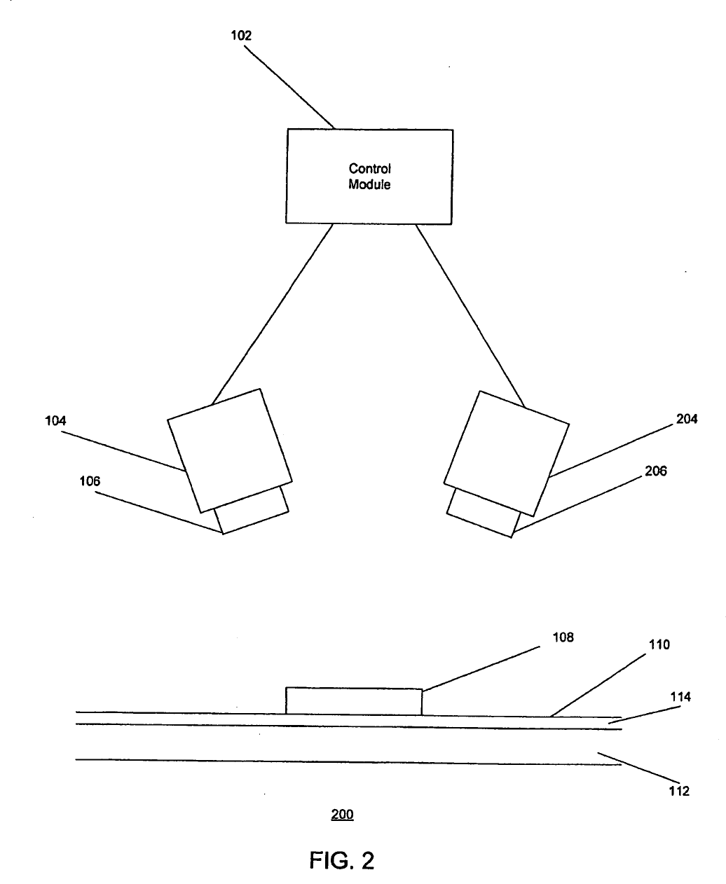

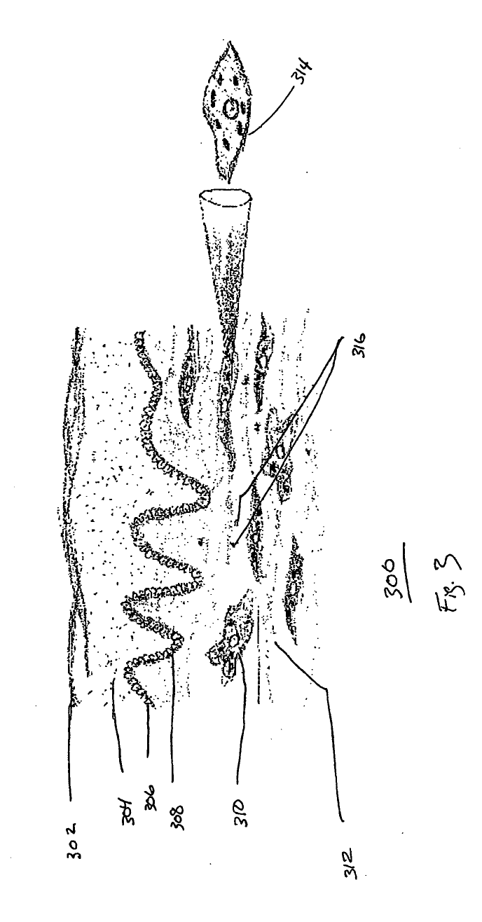

FIG. 2 illustrates a second exemplary embodiment of the dermatological treatment system 200 for conducting various dermatological treatments using EMR to which thermal skin damage of the target area according to the present invention. The system 200 is largely similar to the system 100, except that additional EMR source 204 and delivery optics 206 are provided. As shown in FIG. 2, the system 200 includes the control module 102, the EMR source 104, the delivery optics 106, an EMR source 204, an delivery optics 206 and the optically transparent plate 108. The control module 102 is in communication with the EMR sources 104, 204, which are in turn operatively connected to the delivery optics 106, 206, respectively. In one exemplary variant, the delivery optics 106, 206 can include an optical fiber.

In one exemplary variant of the second embodiment according to the present invention, the control module 102 can be in wireless communication with both the EMR source 104 and the EMR source 204 and/or communication with one or both of the EMR source 104 and the EMR source 204.

The control module 102 provides application specific settings to the EMR sources 104, 204. The EMR sources 104, 204 receive these settings, and generate the EMR based on these settings. Such settings can control the wavelength of the EMR, the energy delivered to the skin, the power delivered to the skin, the pulse duration for each EMR pulse, the fluence of the EMR delivered to the skin, the number of EMR pulses, the delay between individual EMR pulses, the beam profile of the EMR, and the size of the area of the skin exposed to the EMR. The energy produced by the EMR sources 104, 204 can be an optical radiation, which is focused, collimated and/or directed by the delivery optics 106, 206 to the optically transparent plate 108. The optically transparent plate 108 can be placed on a target area of a patient's skin. Prior to the application on the skin, it is preferable to coat the skin with a transparent liquid or gel to provide better optical and thermal coupling between the device and the skin surface. The EMR sources 104, 204 can produce EMR having the same or similar characteristics as well as different characteristics. Preferably, the EMR source 104 and the EMR source 204 may produce the EMR having different wavelengths during the same procedure.

In one exemplary embodiment of the present invention, the EMR source 204 is a laser, a flashlamp, a diode array, a combination of each and the like. In another exemplary embodiment of the present invention, the EMR source 204 is a ruby laser, an alexandrite laser, a neodymium laser, and/or a flashlamp pulsed dye laser.

The system 200 can be used in a manner similar to that of the system 100. The system 200 differs from the system 100 in that the system 200 includes the second EMR source 204. Prior to being used in the dermatological treatment, the system 200 shown in FIG. 2 can be configured by the user. For example, the user may interface with the control module 102 in order to specify the specific settings usable for a particular procedure. The user may specify the wavelength of the EMR, the energy delivered to the skin, the power delivered to the skin, the pulse duration for each EMR pulse, the fluence of the EMR delivered to the skin, the number of the EMR pulses, the delay between individual EMR pulses, the beam profile of the EMR, and the size of the area of skin 110 exposed to the EMR. The EMR sources 104, 204 may be configured to produce a collimated pulsed EMR irradiation with a wavelength between 600 nm and 1200 nm, and between 400 nm and 600 nm, respectively. The pulsed EMR irradiation may be applied which has a pulse duration between 10 μs and 1000 μs, preferably between 5 μs and 200 μs, and ideally the pulse duration is approximately 100 Us, with the fluence being in the range from approximately 0.1 J/cm² to 20 J/cm² (or between 0.1 j/cm² to 40 j/cm²). The applied EMR should be able to achieve a temperature rise within the exposed areas of the skin that is at least sufficient to cause thermal damage to phagocytic cells in the dermis 112.

FIG. 3 illustrates a cross-section of a healthy skin 300 that has been tattooed. The healthy skin 300 includes a stratum corneum 302, an epidermis 304, basal keratinocytes 306, a basement membrane 308, macrophages 310, a dermis 312 and fibroblasts 314. The macrophages 310 and fibroblasts 314 contain tattoo ink due to the application of a tattoo to the skin 300. Extracellular tattoo ink particles 316 may also appear throughout the dermis 312.

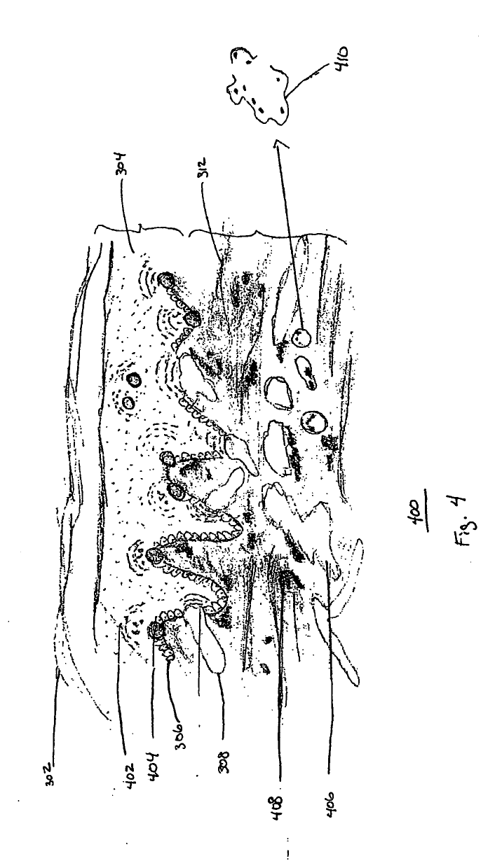

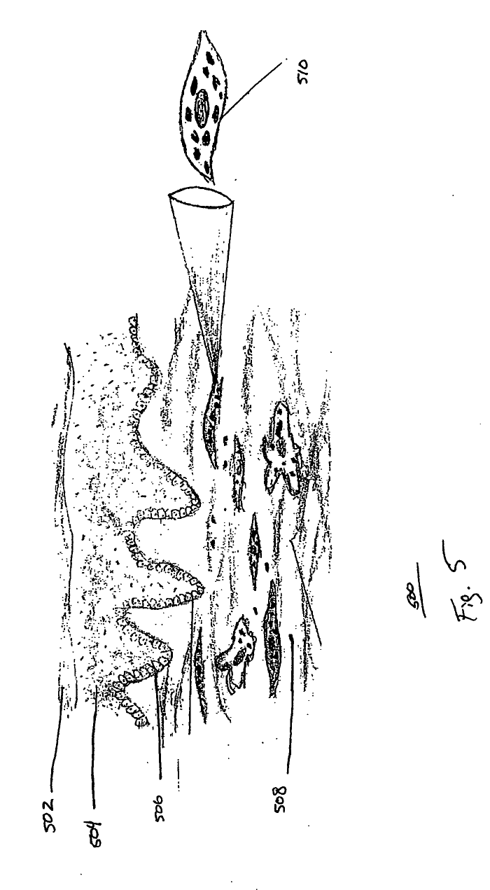

FIG. 4 illustrates a cross-section of skin 400 immediately after a quality switched laser pulse configured for tattoo removal according to conventional techniques has been applied to the skin 400. As shown, the laser pulse caused injury throughout the dermis and the epidermis. The stratum corneum 302 has been disrupted. Stress waves 402 have formed in the target area of the epidermis 304. Throughout the target area, a localized vacuolization 404 of basal keratinocytes 306 has taken place, and the basement membrane 308 has separated from the basal keratinocytes 306. Lacunae 406 have formed in the dermis 312. Also fragmented and scattered tattoo particles 408 can be found throughout the dermis 312, as well as ruptured cells 410 that still contain ink particles. Because certain cells containing ink have ruptured (the ruptured cells 410), inks leaks into the dermis 312, and then it is flushed from the skin through the skin's natural wound healing response over an extended period of time.

FIG. 5 shows a cross-section of skin 500 immediately after an EMR pulse configured for tattoo removal according to the present invention has been applied. The pulse duration range according to an exemplary embodiment of the present invention is approximately one million times longer than that of a Q-switched laser pulse, which results in less unwanted injury, while effectively targeting the phagocytic dermal cells which contain most of the tattoo ink. In sharp contrast to the cross-section of the skin 400 of FIG. 4, the cross-section of the skin 500 shows an intact stratum corneum 502, with no or minimal injury to the epidermis 504, an intact basement membrane 506, a largely healthy dermis 508 and dead or dying fibroblasts 510 containing tattoo ink. Little or no stress waves, vacuolization of basal keratinocytes, separation of the base membrane, and lacunae formation are present, and no or minimal cellular rupture are provided in the cross-section of the skin 500.

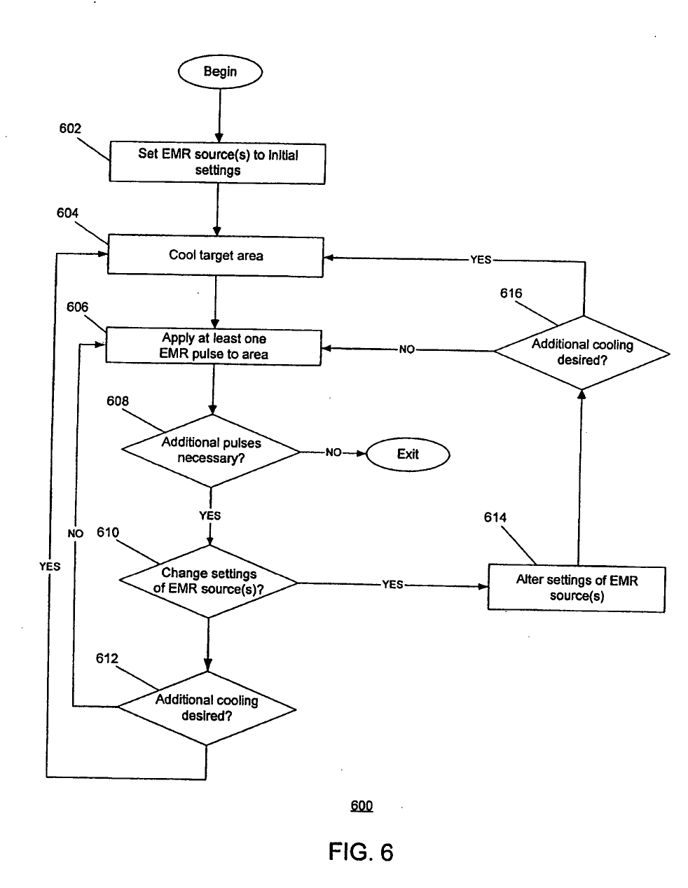

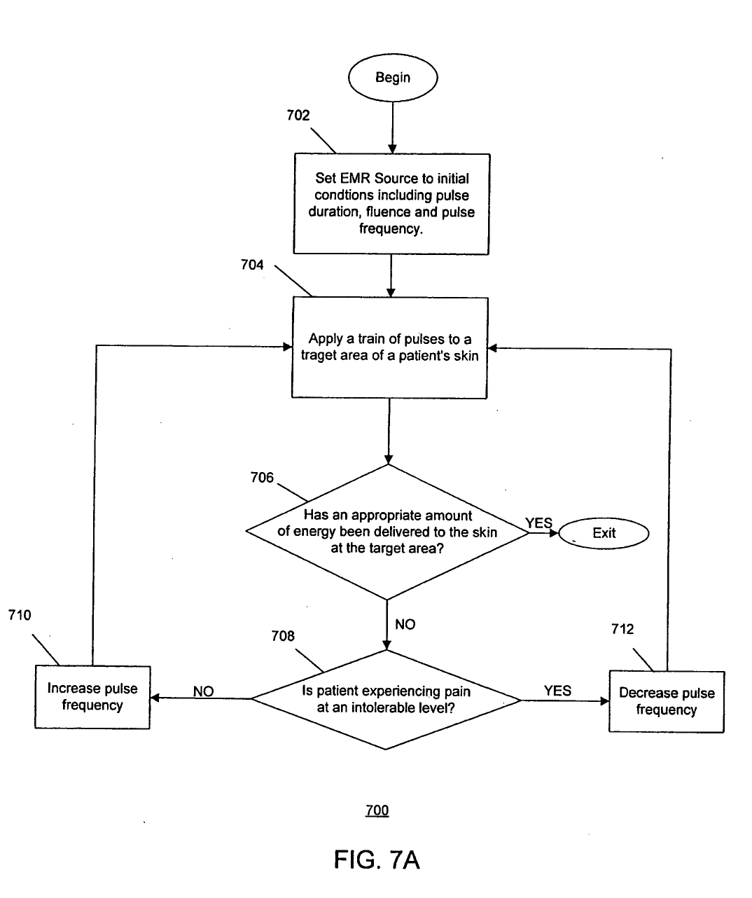

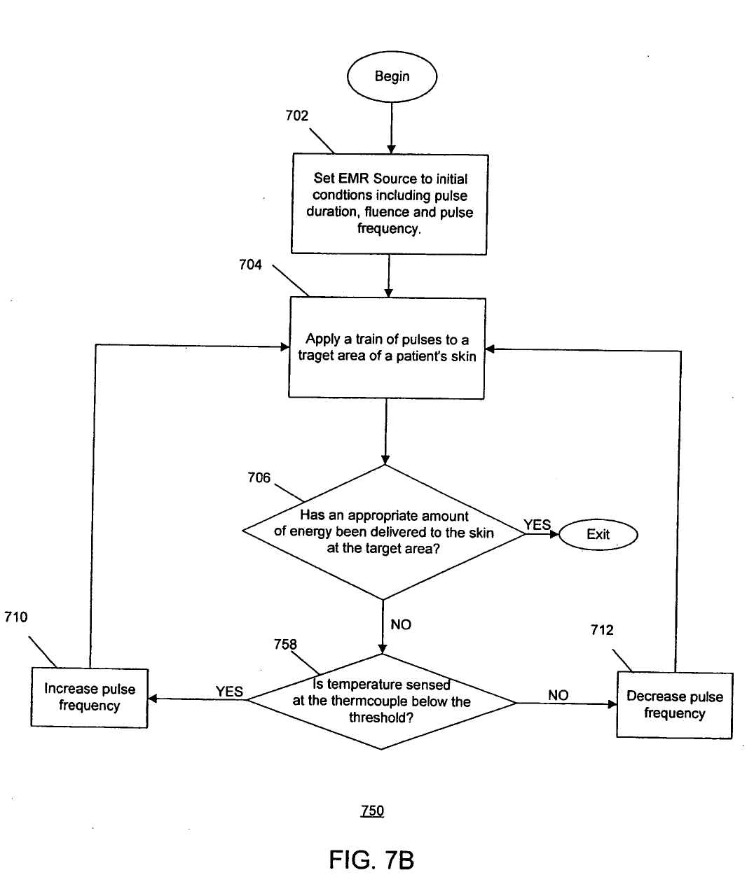

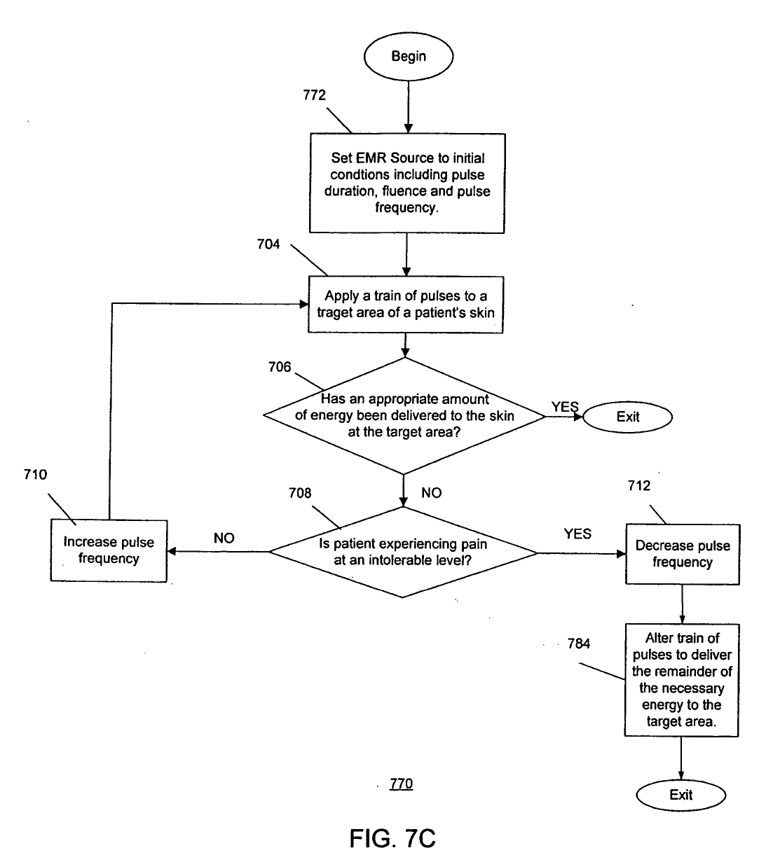

FIG. 6 illustrates a flow chart depicting an exemplary embodiment of a dermatological process 600 using lasers according to the present invention. The process 600 begins at step 602, when the EMR source 104 is set to its initial settings. The EMR source 104 settings can vary widely depending on the type of the dermatological procedure, as well as on the particular problem confronted during the dermatological procedure. For example, the type of dermatological procedure may be tattoo removal. Some of the settings for accomplishing this type of dermatological procedure may be the same for most procedures, however other settings including the wavelength of the EMR used can vary widely, as discussed above, depending on the colors of the particular tattoo to be removed and the EMR source 104, 204 to be used.

In a preferred embodiment of the present invention, the EMR source 204 can be used in conjunction with the EMR source 104. Using the EMR sources 104, 204 in conjunction with each other allows for multiple wavebands to be used at the same time. Different wavebands may target phagocytic cells containing inks of different colors.

At step 604, the target area of the skin may be cooled. Such cooling the target area of the skin assists in preserving the epidermal tissue. The EMR produced by the EMR source 104 may be configured to be minimally absorbed by the epidermis 114; however some of the energy of the EMR emitted by the EMR source 104 is absorbed by the epidermis 114. After cooling the target area of the skin, the process 600 advances to step 606 where at least one EMR pulse is applied to the target area of the skin. The control system 102 specifies the characteristics of each pulse to be applied to the target area, the number of pulses to be applied and the frequency of the pulses. The settings of the control system are highly dependant on the particular procedure being performed at the time. Once the appropriate EMR pulses are applied to the target area, the process 600 can advance to step 608.

In one exemplary embodiment of the present invention, the cooling procedure of step 604 and the application of at least one EMR pulse of step 606 may occur simultaneously. The optically transparent plate 108 can be used to cool the target area of the skin 110. The optically transparent plate 108 can be cooled prior to the procedure or cooled during the procedure. If cooled during the procedure, this is done by circulating a cooling agent through microchannels within the optically transparent plate 108 or by placing a cooling agent adjacent to the optically transparent plate 108.

At step 608, the control system 102 may determine whether additional pulses are necessary to be applied. The number of pulses can be determined before the procedure such that a train of pulses are applied without additional user input during the procedure or during the procedure by the user of the system 100 with the control system 102. If the control system 102 determines that no further EMR pulses are necessary, the process 600 exits. Otherwise, the process 600 advances to step 610, where the control system 102 determines whether a change of the settings of the EMR source 104 is necessary. New settings for the EMR source 104 can be predetermined by the user of the system 100 prior to beginning the procedure or may be determined during the procedure, with the control system 102 by, e.g., pausing after each set of the EMR pulses to await user input. If new settings are not necessary, the process 600 advances to step 612. Otherwise, the process 600 advances to step 614.

At step 612, the control system 102 determines whether additional cooling of the target area is preferable. This cooling step can be set prior to the start of the procedure or can be determined during the procedure by the user of the system 100 with the control system 102, e.g., pausing after each set of EMR pulses to await user input. If additional cooling is necessary, the process 600 advances to step 604. Otherwise, the process 600 advances to step 606.

At step 614, the control system 614 sets the EMR source 104 to appropriate settings. The EMR source 104, 204 settings can vary widely depending on the type of dermatological procedure, as well as the particular problem confronted during the dermatological procedure. Once the EMR source 104, 204 is configured correctly, the process 600 advances to step 616, with which the control system 102 determines whether additional cooling of the target area is necessary. This can be predetermined prior or during the procedure by the user of the system 100 with the control system 102, e.g., again pausing after each set of EMR pulses to await user input. If additional cooling is preferred, the process 600 advances to step 604. Otherwise, the process 600 advances to step 606.

If a flashlamp or alternate intense pulsed light source is used as the EMR source 104, 204, many pulses may be utilized to effectively treat the tattoo. Such a procedure may require, e.g., fifteen minutes (or possibly more) of exposure to the EMR radiation.

FIG. 7A illustrates a dermatological process 700 for using EMR sources according to yet another exemplary embodiment of the present invention to remove and/or diminish the appearance of a tattoo, while not causing the patient an intolerable amount of pain. A temperature rise within the skin may be painful for the patient and is closely related to the amount of EMR delivered to a target area of skin over a particular time period. Delivering a train of pulses, e.g. multiple EMR pulses, to a particular portion of the target area of the skin causes the skin to rise in temperature. Allowing the temperature of the skin to rise above approximately 42° C. may cause the patient to experience pain and/or damage the skin. The actual temperature at which the patient may experience pain and/or damage the skin may be different for various patients. The temperature of the skin may also be regulated by cooling the surface of the skin as shall be described in further detail below.

In particular, the process 700 begins at step 702, such that the EMR source 104 is set to its initial settings. The EMR source 104 can be set or configured to have a particular fluence, pulse duration and pulse frequency. If a flashlamp is used as the EMR source 104, the fluence may be set to be approximately 1000 J/cm², the pulse duration is set to be 1000 μs, and the pulse frequency may be set to be approximately 1 Hz. The EMR source 104 settings may be configured to cause a particular temperature rise in certain structures, including phagocytic cells, within the skin itself. It should be understood that the fluence, pulse duration, EMR wavelength, pulse frequency, and other characteristics of the EMR may be altered to target these structures. Also multiple EMR wavelengths may be used.

As described above, the optically transparent plate 108 is likely also placed on the target area of the patient's skin. Prior to application of the transparent plate 108 on the skin, it is preferable to coat the skin with a transparent liquid or gel to provide better optical and thermal coupling between the plate 108 and the skin surface. The optically transparent place 108 is preferably used to cool the target area as discussed in greater detail above. The optically transparent plate 108 can continuously cool the skin, effectuate the cooling of the skin during application of EMR pulses, or cool the skin between EMR pulses. After the EMR source 104 is configured, the process 700 advances to step 704. In an exemplary embodiment of the present invention, the EMR source 104 can be used in conjunction with the EMR source 204. By using the EMR sources 104, 204 in conjunction with one another, multiple wavebands are capable of being used at the same time. In addition, different wavebands may target phagocytic cells containing inks of different colors.

In step 704, a train of EMR pulses can be applied to a particular portion of the target area of the skin and the optically transparent plate 108 may cool the target area of the skin at the same time. The train of pulses can be applied at a particular frequency defined by a user of the system 100 prior to the start of the procedure. For example, the train of pulses may be applied to the target area for a fixed period of time, until a certain number of pulses have been applied to the target area, and/or until a certain amount of energy has been delivered to the particular portion of the target area. Once the train of pulses has been applied to the target area, the process advances to step 706.

In step 706, the user of the system 100 can determine if an appropriate amount of energy has been applied to the particular portion of the target area. If such amount of energy has been applied to the target area, the procedure may be completed and the process 700 exits. Otherwise, the process 700 advances to step 708.

In step 708, the user of the system 100 determines whether the subject, i.e. the person to whom the EMR is being applied, is experiencing an intolerable amount of pain. If the subject is experiencing such a level of pain, the process 700 advances to step 712 where the pulse frequency may be diminished. Once the pulse frequency is diminished, the process 700 advances to step 704. However, if the subject is not experiencing pain at an intolerable level, the process 700 advances to step 710 where the pulse frequency can be increased. Once the pulse frequency is increased, the process 700 advances to step 704.

FIG. 7B illustrates another exemplary embodiment of a dermatological process 750 according to the present invention for using EMR sources to remove and/or diminish the appearance of a tattoo, while not causing the patient an intolerable amount of pain. The process 750 is substantially identical to the process 700, except that the step 708 is replaced with step 758. Particularly, in step 758, the process 750 may determines whether the temperature of the subject's skin exceeds the temperature threshold (e.g., approximately 42° C.). The temperature of the subject's skin can be measured using a thermocouple affixed to the optically transparent plate 108 and in contact with the skin, a thermocouple in an element of the device which is close to the skin, or a far-infrared detector which monitors black body emission from the skin surface. If the temperature of the subject's skin exceeds the temperature threshold, the process 750 advances to step 712 where the pulse frequency is diminished. Once the pulse frequency is diminished, the process 700 advances to step 704. However, if the temperature of the subject's skin does not exceed the temperature threshold, the process 750 advances to step 710 where the pulse frequency is increased. Once the pulse frequency is increased, the process 750 advances to step 704.

FIG. 7C illustrates a dermatological process 770 according to still another exemplary embodiment of the present invention for using EMR sources to remove and/or diminish the appearance of a tattoo, while not causing the patient an intolerable amount of pain. The process 770 is substantially identical to the process 700, except that the step 702 is replaced with step 772, and step 712 is followed by step 784.

The process 770 begins at step 772 where the EMR source 104 is set to its initial settings in approximately the same manner as described above in relation to the process 702, except that the pulse frequency can be set extremely low. The pulse frequency may be set at a rate that is below the rate, such that it would be possible for the subject to experience an intolerable amount of pain, for example, the amount of EMR delivered to the target area of the skin cannot overcome the cooling effect of the optically transparent plate 108.

In step 712, after the user decreased the pulse frequency, the process 770 advances to step 784. In step 784, the user may alter the train of pulses to be applied to the particular portion of the target area. From the beginning of the process 770, the pulse frequency of the train of pulses may have been gradually increased until the subject's pain tolerance has been reached. Following this gradual increase of the pulse frequency, the pulse frequency diminished such that the subject does not experience the intolerable amount of pain while the train of pulses is being applied to the target area. Thus, an equilibrium has been attained the train of pulses increases the temperature of the subject's skin, while the optically transparent plate 108 cools the target area of the subject's skin. Since this equilibrium has been attained, the user may alter the train of pulses to deliver the remainder of the necessary pulses, can apply the train of pulses to the particular portion of the target area of the subject's skin, and the process 770 exits. This may result in a longer train of pulses, however, since the equilibrium has been attained, the patient will likely not experience an intolerable pain.

The foregoing merely illustrates the principles of the invention. Various modifications and alterations to the described embodiments will be apparent to those skilled in the art in view of the teachings herein. It will thus be appreciated that those skilled in the art will be able to devise numerous techniques which, although not explicitly described herein, embody the principles of the invention and are thus within the spirit and scope of the invention.

1. A light emitting apparatus, comprising:
a laser-emitting arrangement specifically configured to produce particular radiation pulses that target phagocytic cells containing at least one of particles of melanin and exogenous artificial pigment when skin of a subject is impinged by the particular radiation, wherein the particular radiation has a fluence range between 1 J/cm² and 20 J/cm² and a pulse width of at least 1 μs in duration and at most 300 μs in duration.

2. The light emitting apparatus of claim 1, wherein the particular radiation has a fluence range between 5 J/cm² and 10 J/cm².

3. The light emitting apparatus of claim 1, wherein the particular radiation has a spot-size diameter of the particular radiation beam of at least 3 mm.

4. The light emitting apparatus of claim 1, wherein the particular radiation has a spot-size diameter of at least 10 mm.

5. The light emitting apparatus of claim 1, wherein the pulses are emitted at a frequency of between 1 Hz and 100 Hz.

6. The light emitting apparatus of claim 1, wherein the pulses are emitted at a frequency of approximately 10 Hz.

7. The light emitting apparatus of claim 1, wherein the particular radiation has a waveband approximately equal to that of blue light.

8. The light emitting apparatus of claim 7, wherein the waveband is between approximately 400 nm and 600 nm.

9. The light emitting apparatus of claim 7, wherein the waveband is between approximately 400 nm and 550 nm.

10. The light emitting apparatus of claim 1, wherein the particular radiation has a waveband approximately equal to that of broadband red-near infrared light.

11. The light emitting apparatus of claim 10, wherein the waveband is between approximately 600 nm and 1200 nm.

12. The light emitting apparatus of claim 1, wherein the optical radiation has a pulse width of at least 50 μs in duration and at most 200 μs in duration.

13. The light emitting apparatus of claim 1, wherein the laser-emitting arrangement is one of a ruby laser, an alexandrite laser, a neodymium laser, and a flashlamp-pumped pulsed dye laser.

14. A light emitting apparatus, comprising:
a radiation generator configured to produce particular radiation pulses, each of which have a fluence range between approximately 2 J/cm² and 20 J/cm² and a pulse width of between 1 μs and 300 μs in duration, wherein the particular radiation pulses target a portion of a target area, and wherein the particular radiation pulses are emitted at a frequency of between 1 Hz and 20 Hz.

15. The light emitting apparatus of claim 14, wherein the radiation generator is configured to product a spot-size diameter of the particular radiation beam of at least 3 mm.

16. The light emitting apparatus of claim 14, wherein the fluence range of the particular radiation is between 5 J/cm² and 10 J/cm².

17. The light emitting apparatus of claim 14, wherein the spot-size diameter of the particular radiation is at least 10 mm.

18. The light emitting apparatus of claim 14, wherein the particular radiation has a pulse width between 50 μs and 200 μs in duration.

19. The light emitting apparatus of claim 14, wherein the radiation generator is a laser-emitting arrangement.

20. The light emitting apparatus of claim 19, wherein the laser is one of a ruby laser, an alexandrite laser, a neodymium laser, and a flashlamp-pumped pulsed dye laser.

21. A method for decreasing the appearance of a tattoo on tattooed dermal tissue, comprising:
generating particular radiation using a laser-emitting arrangement having a fluence range between approximately 1 J/cm² and 20 J/cm², a spot-size diameter of the particular radiation beam of at least 3 mm, and a pulse width of between 1 its and 300 μs in duration; and
exposing the dermal tissue of a subject to the particular radiation.

22. The method of claim 21, wherein the radiation generator is a laser.

23. The method of claim 22, wherein the laser-emitting arrangement is one of a ruby laser, an alexandrite laser, a neodymium laser, and a flashlamp-pumped pulsed dye laser.

24. A method for decreasing the appearance of a tattoo on tattooed dermal tissue, comprising:
generating particular radiation using a laser-emitting arrangement that targets phagocytic cells when the dermal tissue of a subject is exposed to the particular radiation, wherein the particular radiation having a fluence range between approximately 1 J/cm² and 20 J/cm² and a pulse width of between 1 its and 300 μs in duration; and
exposing the skin tissue of the subject to the particular radiation.

25. The method of claim 24, wherein the particular radiation having a spot-size diameter of the particular radiation beam of at least 3 mm.

26. The method of claim 24, wherein the particular radiation is generated by a laser.

27. The method of claim 24, wherein the laser is one of a ruby laser, an alexandrite laser, a neodymium laser, and a flashlamp-pumped pulsed dye laser.

28. A method for decreasing the appearance of a tattoo on tattooed dermal tissue, comprising:
(a) generating a plurality of radiation pulses specifically adapted to target phagocytic cells when the dermal tissue of a subject is exposed to the particular radiation, wherein the plurality of radiation pulses having a fluence range between approximately 1 J/cm² and 20 J/cm² and a pulse width of between 1 μs and 300 μs in duration;
(b) exposing the skin tissue of the subject to the radiation pulses at a particular frequency;
(c) determining whether the subject is at least one of experiencing and has experienced pain; and
(d) during step (d), based on a result of step (c), controlling the particular frequency.

29. The method of claim 28, wherein in step (d), the frequency is increased if the subject does not experience pain.

30. The method of claim 28, wherein in step (d), the frequency is decreased if the subject experiences pain.

31. The method of claim 28, wherein the plurality of radiation pulses having a spot-size diameter of the radiation beam of at least 3 mm.

32. The method of claim 28, wherein the plurality of radiation pulses is generated by a laser-emitting arrangement.

33. The method of claim 32, wherein the laser is one of a ruby laser, an alexandrite laser, a neodymium laser, and a flashlamp-pumped pulsed dye laser.

34. A method for decreasing the appearance of a tattoo on tattooed dermal tissue, comprising:
(a) generating a plurality of radiation pulses specifically adapted to target phagocytic cells when the dermal tissue of a subject is exposed to the particular radiation, wherein the plurality of radiation pulses having a fluence range between approximately 1 J/cm² and 20 J/cm² and a pulse width of between 1 μs and 300 μs in duration;
(b) exposing the skin tissue of the subject to the radiation pulses at a particular frequency;
(c) determining whether the temperature of the skin exceeds a threshold value; and
(d) during step (d), based on a result of step (c), controlling the particular frequency.

35. The method of claim 34, wherein in step (d), the frequency is increased if the threshold value is not exceeded.

36. The method of claim 34, wherein in step (d), the frequency is decreased if the threshold value is met or exceeded.

37. The method of claim 34 wherein the threshold value is 42 degrees Centigrade.

38. The method of claim 34, wherein the plurality of radiation pulses having a spot-size diameter of the radiation beam of at least 3 mm.

39. The method of claim 34, wherein the plurality of radiation pulses is generated by a laser-emitting arrangement.

40. The method of claim 39, wherein the laser-emitting arrangement is one of a ruby laser, an alexandrite laser, a neodymium laser, and a flashlamp-pumped pulsed dye laser.

41. A light emitting apparatus, comprising a radiation generator specifically configured to produce a plurality of particular radiation pulses that target phagocytic cells containing at least one of particles of melanin and exogenous artificial pigment when skin of a subject is impinged by the particular radiation, wherein the particular radiation has a fluence range between 0.1 J/cm² and 20 J/cm² and a pulse width of at least 10 μs in duration and at most 1000 μs in duration, and wherein the plurality of pulses are applied to a particular portion of a target area at a rate of at least 1 Hz and at most 100 Hz.

42. The light emitting apparatus of claim 41, wherein the particular radiation has a waveband approximately equal to that of blue light.

43. The light emitting apparatus of claim 42, wherein the waveband is between approximately 400 nm and 600 nm.

44. The light emitting apparatus of claim 42, wherein the waveband is between approximately 400 nm and 550 nm.

45. The light emitting apparatus of claim 41, wherein the particular radiation has a waveband approximately equal to that of green light.

46. The light emitting apparatus of claim 45, wherein the waveband is between approximately 500 nm and 600 nm.

47. The light emitting apparatus of claim 41, wherein the particular radiation has a waveband approximately equal to that of broadband red-near infrared light.

48. The light emitting apparatus of claim 47, wherein the waveband is between approximately 600 nm and 1200 nm.

49. The light emitting apparatus of claim 41, wherein the particular radiation has a fluence range between 0.1 J/cm² and 1 J/cm².

50. The light emitting apparatus of claim 41, wherein the particular radiation has a spot-size diameter of the particular radiation beam of at least 3 mm.

51. The light emitting apparatus of claim 41, wherein the particular radiation has a spot-size diameter of at least 10 mm.

52. The light emitting apparatus of claim 41, wherein the particular radiation has a spectral bandwidth of at least 50 nm.

53. The light emitting apparatus of claim 41 wherein the particular radiation has a spectral bandwidth of at least 100 nm.

54. The light emitting apparatus of claim 41, wherein the particular radiation has a spectral bandwidth of at least 100 nm and at most 500 nm.

55. The light emitting apparatus of claim 41, wherein the optical radiation has a pulse width of at least 50 μs in duration and at most 200 μs in duration.

56. The light emitting apparatus of claim 41, wherein the optical radiation has a pulse width of at least 10 μs in duration and at most 50 μs in duration.

57. The light emitting apparatus of claim 41, wherein the optical radiation has a pulse width of at least 200 μs in duration and at most 1000 μs in duration.

58. The light emitting apparatus of claim 41, wherein the radiation generator is one of a flashlamp, a tungsten lamp, a diode, an arc lamp, a laser diode array, and a diode array.

59. The light emitting apparatus of claim 41, wherein the radiation generator is one of a Xenon flashlamp, a mixed gas flashlamp and a doped flashlamp.

60. The light emitting apparatus of claim 41, further comprising:
a temperature sensing devise configured to sense a temperature of the particular position of the target area of skin.

61. The light emitting apparatus of claim 60, further comprising:
a control device configured to receive the temperature sensed by the temperature sensing device and alter certain of the plurality of the particular radiation pluses based at least in part upon the sensed temperature.

62. A method for decreasing the appearance of a tattoo on tattooed dermal tissue, comprising:
(a) generating a plurality of radiation pulses specifically adapted to target phagocytic cells when the dermal tissue of a subject is exposed to the particular radiation, wherein the radiation pulses have a fluence range between approximately 0.1 J/cm² and 20 J/cm² and a pulse width of between 10 μs and 1000 μs in duration;
(b) exposing the skin tissue of the subject to the radiation pulses at a particular frequency;
(c) determining whether the subject is at least one of experiencing and has experienced pain; and
(d) during step (d), based on a result of step (c), controlling the particular frequency.

63. The method of claim 62, wherein in step (d), the frequency is increased if the subject does not experience pain.

64. The method of claim 62, wherein in step (d), the frequency is decreased if the subject experiences pain.

65. The method of claim 62, wherein the plurality of radiation pulses having a spot-size diameter of the radiation beam of at least 3 mm.

66. The method of claim 62, wherein the plurality of radiation pulses is generated by a flashlamp.

67. The method of claim 66, wherein the flashlamp is one of a Xenon flashlamp; a mixed gas flashlamp, and a doped flashlamp.

68. A method for decreasing the appearance of a tattoo on tattooed dermal tissue, comprising:
(a) generating a plurality of radiation pulses specifically adapted to target phagocytic cells when the dermal tissue of a subject is exposed to the particular radiation, wherein the radiation pulses have a fluence range between approximately 0.1 J/cm² and 20 J/cm² and a pulse width of between 10 μs and 1000 μs in duration;
(b) exposing the skin tissue of the subject to the radiation pulses at a particular frequency;
(c) determining whether the temperature of the skin exceeds a threshold value; and
(d) during step (d), based on a result of step (c), controlling the particular frequency.

69. The method of claim 68, wherein in step (d), the frequency is increased if the threshold value is not exceeded.

70. The method of claim 68, wherein in step (d), the frequency is decreased if the threshold value is met or exceeded.

71. The method of claim 68, wherein the threshold value is 42 degrees Centigrade.

72. The method of claim 68, wherein the plurality of radiation pulses having a spot-size diameter of the radiation beam of at least 3 mm.

73. The method of claim 68, wherein the plurality of radiation pulses is generated by a flashlamp.

74. The method of claim 68, wherein the flashlamp is one of a Xenon flashlamp, a mixed gas flashlamp and a doped flashlamp.

75. A light emitting apparatus, comprising a laser producing radiation that affects phagocytic cells in a target portion of skin, the phagocytic cells including at least one of a particle of melanin and a particle of an exogenous artificial pigment.

76. The light emitting apparatus of claim 75, wherein the laser produces radiation comprises a fluence of between about 0.1 J/cm² and about 40 J/cm².

77. The light emitting apparatus of claim 75, wherein the laser produces radiation comprising a wavelength of about 532 nm, a pulse rate of between about 1 Hz and 3 Hz, and a pulse duration of about 100 ms.

78. The light emitting apparatus of claim 75, wherein the laser produces radiation comprising a wavelength of about 755 nm, a pulse rate of between about 1 Hz and 3 Hz, and a pulse duration of about 100 ms.

79. The light emitting apparatus of claim 75, wherein the laser produces radiation comprising a wavelength of about 1064 nm, a pulse rate of between about 1 Hz and 5 Hz, and a pulse duration of about 120 ms.

80. The light emitting apparatus of claim 75, further comprising a plurality of laser sources, each laser source producing radiation with a different wavelength.

81. The light emitting apparatus of claim 80, wherein the plurality of laser sources comprise a first laser source having a wavelength of about 532 nm and a second laser source having a wavelength of about 755 nm.

82. The light emitting apparatus of claim 81, wherein the plurality of laser sources further comprise a third laser source having a wavelength of about 1064 nm.

83. A method of improving the appearance of a skin marking including an exogenous artificial pigment, comprising:
providing a beam of radiation produced by a laser; and
thermally damaging phagocytic cells by delivering the beam of radiation, the phagocytic cells including at least one particle of an exogenous artificial pigment.

84. The method of claim 83, further comprising providing a beam of radiation having a fluence between 0.1 J/cm² and 40 J/cm².

85. The method of claim 83, wherein the beam of radiation comprises a wavelength of about 532 nm, a pulse rate of between about 1 Hz and 3 Hz, and a pulse duration of about 100 ms.

86. The method of claim 83, wherein the beam of radiation comprises a wavelength of about 755 nm, a pulse rate of between about 1 Hz and 3 Hz, and a pulse duration of about 100 ms.

87. The method of claim 83, wherein the beam of radiation comprises a wavelength of about 1064 nm, a pulse rate of between about 1 Hz and 5 Hz, and a pulse duration of about 120 ms.

88. The method of claim 83, further comprising providing radiation comprising a plurality of wavelengths.

89. The method of claim 88, wherein the beam of radiation comprises a first wavelength of about 532 nm and a second wavelength of about 755 nm.

90. The method of claim 89, wherein the beam of radiation further comprises a third wavelength of about 1064 nm.

91. A light emitting apparatus, comprising:
a laser-emitting arrangement specifically configured to produce a series of particular radiation pulses that target phagocytic cells containing at least one of particles of melanin and exogenous artificial pigment when skin of a subject is impinged by the particular radiation.

92. A method for decreasing the appearance of a tattoo on tattooed dermal tissue, comprising:
generating a series of particular radiation pulse using a laser-emitting arrangement that targets phagocytic cells when the dermal tissue of a subject is exposed to the particular radiation; and
exposing the skin tissue of the subject to the particular radiation.

93. A light emitting apparatus, comprising a radiation generator specifically configured to produce a plurality of particular radiation pulses that target phagocytic cells containing at least one of particles of melanin and exogenous artificial pigment when skin of a subject is impinged by the particular radiation, wherein the particular radiation has a fluence range between 0.1 J/cm² and 40 J/cm² and a pulse width of at least 10 μs in duration and at most 1000 μs in duration, and wherein the plurality of pulses are applied to a particular portion of a target area at a rate of at least 1 Hz and at most 100 Hz.

94. A method for decreasing the appearance of a tattoo on tattooed dermal tissue, comprising:
(a) generating a plurality of radiation pulses specifically adapted to target phagocytic cells when the dermal tissue of a subject is exposed to the particular radiation, wherein the radiation pulses have a fluence range between approximately 0.1 J/cm² and 40 J/cm² and a pulse width of between 10 μs and 1000 μs in duration;
(b) exposing the skin tissue of the subject to the radiation pulses at a particular frequency;
(c) determining whether the subject is at least one of experiencing and has experienced pain; and
(d) during step (d), based on a result of step (c), controlling the particular frequency.
